(19) United States
(12) Patent Application Publication
(10) Pub. No.: US 2014/0377756 A1
(43) Pub. Date: Dec. 25, 2014

(54) METHOD FOR QUANTIFYING SKIN CHANGES CAUSED BY SIX EXTERNAL EVILS AND METHOD FOR SCREENING SKIN CONDITION-IMPROVING MATERIALS USING THE SAME

(71) Applicant: AMOREPACIFIC CORPORATION, Seoul (KR)

(72) Inventors: Jin Young LEE, Seoul (KR); Sun Sang KWON, Yongin (KR); Myeong Hoon YEOM, Yongin (KR); Duck Hee KIM, Seoul (KR); Han Kon KIM, Suwon (KR)

(21) Appl. No.: 14/478,733

(22) Filed: Sep. 5, 2014

Related U.S. Application Data
(63) Continuation of application No. 13/128,697, filed on May 11, 2011.

(30) Foreign Application Priority Data
Nov. 11, 2008 (KR) 10-2008-0111838

(51) Int. Cl. G01N 33/53; C12Q 1/02; C12Q 1/30; C12Q 1/68; C12Q 1/32

(52) U.S. Cl. CPC G01N 33/53; C12Q 1/68; C12Q 1/686; C12Q 1/32; C12Q 1/30; C12Q 1/02; G01N 2500/04
USPC 435/6.11; 435/6.12; 435/7.1; 435/7.92; 435/26; 435/27; 435/29

(57) ABSTRACT

A method for quantifying skin changes caused by six external evils and a method of screening skin condition-improving materials using the quantification method are described. More specifically, disclosed are a method of measuring cellular changes caused by external stimuli in a skin cell culture system, in which the degree of cellular changes obtained by applying suitable stimuli of six external evils to skin cells being cultured is measured by cellular biochemical methods, such that the conceptual effects of six external evils suggested in the prior art can be scientifically and quantitatively expressed, and a method of screening skin condition-improving materials using the measurement method.

### CROSS-REFERENCE TO RELATED APPLICATIONS

This application is a continuation of application Ser. No. 13/128,697, filed May 11, 2011, pending; which is the U.S. national phase of Application No. PCT/KR2009/006480, filed Nov. 5, 2009; which designated the U.S. and claims priority to KR 10-2008-0111838, filed Nov. 11, 2008; the entire contents of each of which are hereby incorporated by reference.

### TECHNICAL FIELD

The present invention relates to a method for quantifying skin changes caused by six external evils and a method of screening skin condition-improving materials using the quantification method, and more particularly to a method of measuring cellular changes caused by external stimuli in a skin cell culture system, in which the degree of cellular changes obtained by applying suitable stimuli of six external evils to skin cells being cultured is measured by cellular biochemical methods, such that the conceptual effects of six external evils suggested in the prior art can be scientifically and quantitatively expressed, and to a method of screening skin condition-improving materials using the measurement method.

### BACKGROUND ART

In Chinese herbal medicine, “six external evils” refer to factors having adverse effects on the human body, that is, the causes of diseases, and commonly refer to external pathogenic factors, including wind, cold, heat, dampness, dryness and fire. Generally, wind, cold, heat, dampness, dryness and fire are six different climatic changes in nature and are called “six atmospheric influences”. The six atmospheric influences are conditions in which everything grows. The six atmospheric influences are harmless to the human body in their normal states. However, if the climatic changes are abnormal and the generation of the six atmospheric influences is excessive or insufficient, the six atmospheric influences become disease-causing factors and invade the human body to cause diseases. In this case, the six atmospheric influences are called the “six excesses or six evils”.

The six excesses may cause changes mainly in the conditions of the face and skin regions. Because the face region is always exposed to an external environment, it must withstand changes in external conditions, including wind, cold and heat. The skin acts as a first defense barrier to protect the human body from invasion of the six excesses. If the six excesses invade the human body, they will cause skin aging, and particularly, severe cold, severe heat, dryness and strong sunlight will cause very severe damage to the skin. The physiological aging phenomenon of the skin has a close connection with the six excesses in the human living environment. The six excesses act as the external causes of skin aging phenomena to directly cause various skin diseases and five sensory organ diseases or to make diseases worse.

In the cosmetic field, a variety of herbal cosmetic products have been developed, and in addition, various herbal theories have been applied in the manufacture of cosmetic products. However, even if the concept of the six excesses was used to manufacture herbal cosmetic products, the effects of the materials of the herbal cosmetic products could not be scientifically and objectively proven.

### DISCLOSURE OF INVENTION

### Technical Problem

Accordingly, the present inventors have visualized and quantified external stimuli corresponding to the six excesses and skin cell changes caused by the external stimuli using a skin cell culture system through cellular biochemical technology, thereby developing a method of screening materials capable of defending the skin from the six external evils, thereby completing the present invention.

It is, therefore, an object of the present invention to construct a skin cell culture system capable of scientifically expressing skin changes caused by the six external evils and to provide a method of quantifying skin changes caused by the six external evils through the constructed cell culture system and a method of screening skin condition-improving materials using the quantification method.

### Solution to Problem

To achieve the above object, the present invention provides a method for quantifying skin changes caused by six external evils, the method comprising the steps of:
(a) selecting suitable skin stimuli of six external evils, including wind, cold, heat, dampness, dryness and fire evils;
(b) determining a cellular biochemical method, which can measure skin cell changes, according to each of the stimuli;
(c) measuring the condition of the skin of a subject by the biochemical method;
(d) stimulating the skin with each of the stimuli, and then quantifying the degree of cellular changes according to the intensity of the stimuli; and
(e) applying medicinal herbs, which defend the skin from the six external evils, to the skin, and then measuring and quantifying the conditions of the skin by the biochemical method.

The present invention also provides a method of screening a skin condition-improving material using said method for quantifying skin changes.

### Advantageous Effects of Invention

The inventive method for quantifying skin changes caused by the six external evils can scientifically and quantitatively express skin changes after applying stimuli corresponding to the six external evils in a skin cell culture system. The results of this quantitative assay can be reconfirmed through the effects of medicinal herbs corresponding to the applied stimuli.

Accordingly, in discovering materials that can be used to manufacture herbal cosmetic products, the herbal effects of the materials can be objectively expressed through scientific results, thus increasing the effects of the cosmetic products. Also, this method can be broadly applied to a method of obtaining a skin sample from a user of a cosmetic product and measuring a change in the skin condition of the user.

### BEST MODE FOR CARRYING OUT THE INVENTION

The present invention is directed to a method for quantifying skin changes caused by six external evils, the method comprising the steps of:
(a) selecting suitable skin stimuli of six external evils, including wind, cold, heat, dampness, dryness and fire evils;
(b) determining a cellular biochemical method, which can measure skin cell changes, according to each of the stimuli;
(c) measuring the condition of the skin of a subject by the biochemical method;
(d) stimulating the skin with each of the stimuli, and then quantifying the degree of cellular changes according to the intensity of the stimuli; and
(e) applying medicinal herbs, which defend the skin from the six external evils, to the skin, and then measuring and quantifying the conditions of the skin by the biochemical method.

As used herein, the term “six external evils” refers to six external factors, including wind, cold, heat, dampness, dryness and fire evils, which stimulate the skin to cause skin aging.

As used herein, the phrase “skin changes caused by the six external evils” is meant to include general skin changes between before and after exposure to the six external evils. Preferably, the phrase means skin damages caused by the six external evils, and examples of the skin damages include, but are not limited to, skin aging, pigmentation, skin dryness and skin troubles.

As used herein, the term “skin condition-improving material” refers to a material having the effect of improving the skin aging, pigmentation, skin dryness or skin troubles caused by the six external evils.

Hereinafter, each step of the inventive method for quantifying skin changes caused by the six external evils will be described in further detail.

First, suitable stimuli of the six external evils are selected. In the present invention, on the basis of the concept of Chinese medicinal books such as Donguibogam, an inflammation-inducing factor was selected as the wind stimulus, and a low-temperature stimulus was selected for cold evil, a high-temperature stimulus for heat evil, active oxygen species (ROS) for dampness evil, and an ultraviolet light stimulus for fire evil. The selected stimuli will now be described in further detail.

1) Wind evil: Stimuli corresponding to wind evil are factors that can cause inflammations on the skin. Examples of the inflammation-inducing factors which can be used in the present invention include atmospheric pollutants such as formaldehyde, allergic materials such as ticks and pollens, yellow sand, bacterial lipopolysaccharide (LPS), phorbol myristate acetate (PMA), and inflammation-inducing cytokines. In the present invention, the inflammation-inducing factors may be used in an amount of 0.0001-10 wt % based on the weight of a test sample (or cultured cells). If the amount of inflammation-inducing factors used is less than 0.0001 wt %, a stimulus having a suitable intensity cannot be induced, and if it exceeds 10 wt %, the intensity of the stimulus will be too strong such that all the cells are killed.

The method of quantifying skin changes through a cellular reaction that uses wind evil as a stimulus as described above can measure skin changes by measuring either the change in production of TNF-α and PGE₂ or the change in production or activity of COX-2 protein, which induces TNF-α and PGE₂, through any conventional method well known in the art. In addition, inflammation-related or skin-related biological indices may also be properly used.

2) Cold evil: A stimulus corresponding to cold evil is a cold stimulus which can cause damage to the skin. The cold stimulus is set at a temperature lower than the temperature of the skin. The set temperature may range from room temperature (about 35° C.) to low temperature (about −40° C.). If the set temperature is lower than −40° C., the intensity of the stimulus will be too strong such that all the cells are killed, and if it is higher than 35° C., it will not be meaningful as the cold stimulus, because it is within the normal body temperature range. The method of quantifying skin changes through a cellular reaction that uses cold evil as a stimulus as described above can measure skin changes by properly using the change in cell proliferation rate, the change in cell metabolism and other skin-related biological indices, which are conventionally used in the art.

3) Heat evil: A stimulus corresponding to heat evil is a heat stimulus that can cause damage to the skin. The heat stimulus is set at a temperature higher than the temperature of the skin. The set temperature may range from 39° C. to 50° C. If the set temperature is lower than 39° C., it will not be meaningful as the heat stimulus, because it is within the normal body temperature range, and if it higher than 50° C., the intensity of the stimulus will be too strong such that all the cells are killed. The method of quantifying skin changes through a cellular reaction that uses heat as a stimulus as described above can measure skin changes by properly using the change in cell damage, the change in cell metabolism, and other skin-related biological indices, which are conventionally used in the art.

4) Dampness evil: In the present invention, it is considered that, because skin damage caused by dampness evil occurs mainly due to the accumulation of an impure and dirty matter in the rainy reason, circumstances of strong dampness are related to that the impure and dirty matter is accumulated on the skin to increase the amount of reactive oxygen species which are produced as by-products of energy metabolism. For this reason, a reactive oxygen species stimulus was selected as a stimulus corresponding to dampness evil. The reactive oxygen species stimuli include not only directly treating reactive oxygen species, but also treating materials or stimuli which induce reactive oxygen species in vivo. For example, the reactive oxygen species stimuli may include materials such as FeCl₃ or FeCl₂, which can induce reactive oxygen radicals by the Fenton reaction. In one Example of the present invention, an H₂O₂ stimulus was used, but the scope of the present invention is not limited thereto.

The H₂O₂ stimulus is preferably carried out by applying H₂O₂ at a dose of 0.001-10 mM. If the dose is less than 0.001 mM, the intensity of the stimulus will be low so as to make it difficult to compare with a normal state. On the other hand, if the dose exceeds 10 mM, the intensity of the stimulus will be too strong such that all the cells are killed.

The method of quantifying skin changes through a cellular reaction that uses dampness evil as a stimulus as described above can measure skin changes by properly using the change in cell damage, the change in a cellular antioxidant system and other skin-related biological indices, which are conventionally used in the art.

5) Dry evil: A stimulus corresponding to dry evil is a dry stimulus that can cause damage to the skin. The dry stimulus includes not only exposing the skin directly to dry air, but also treating materials or stimuli that induce reactions similar thereto in vivo. As used herein, the term “dry air” means an air having a moisture content of less than 60%. If a dry air having a moisture content of 35% is used as a stimulus, the exposure time may be limited to between 5 minutes and 20 minutes. If the exposure time is less than 5 minutes, the intensity of the dry stimulus will be low, and if it exceeds 20 minutes, the intensity of the stimulus will be too strong such that all the cells are killed. However, specific experimental conditions (moisture content, exposure time, etc.) may be easily controlled by a person skilled in the art depending on the measurement of the indoor humidity of an experimental place.

The method of quantifying skin changes through a cellular reaction that uses dry evil as a stimulus as described above can measure skin changes by properly using the change in cell damage, the change in cell proliferation rate, the change in an in vivo calcium signaling system and other skin-related biological indices, which are conventionally used in the art.

6) Fire evil: A stimulus corresponding to fire evil may be a sunlight stimulus that can cause damage to the skin. Examples of the sunlight stimulus include an UV-B stimulus, an UV-A stimulus, a visible stimulus and an IR stimulus. The sunlight stimulus is preferably carried out by radiating sunlight at a dose of 5-200 mJ/cm². If the dose is less than 5 mJ/cm², the intensity of the stimulus will be low so as to make it difficult to compare with a normal state, and if it exceeds 200 mJ/cm², the intensity of the stimulus will be too strong such that all the cells are killed.

The method of quantifying skin changes through a cellular reaction that uses fire devil as a stimulus as described above can measure skin changes by properly using the change in cell damage, the change in the body's defense system and other skin-related biological indices, which are conventionally used in the art.

The above-described six stimuli correspond to the six external evils, and in the case of the six internal evils, other mechanisms and stimuli may be applied.

The six external evils described in the present invention can invade the body alone, and two or more of the external evils can simultaneously invade the body and may be converted into other types of evils. Thus, suitable biological indices and suitable typical medicinal herbs may be applied for the six external evils. For example, wind evil may invade the body and change to heat evil (referred to as “wind-heat evil”), and cold may change to dampness (referred to as “cold-dampness evil”).

Then, the intensity of each of the stimuli and a suitable cell biochemical method for each of the stimuli are determined. This determination step is carried out using a cellular biochemical method that can be characteristically used depending on the kind of stimulus described in the selection step. By confirming that the cellular reactions are alleviated or inhibited by typical medicinal herbs and herbal prescriptions corresponding thereto that restore changes caused by the six external evils (i.e., wind, cold, heat, dampness, dryness and fire evils), skin aging caused by wind evil can be determined through a significant difference in the changes.

The cellular biochemical methods which are carried out for quantification may include all conventional methods which are well known in the art. For example, the cellular biochemical methods include: TNF-α ELISA, PGE₂ ELISA or interleukin expression analysis for quantification of skin changes caused by wind evil; cell proliferation assay or FAS analysis for quantification of skin changes caused by cold evil; RT-PCR or immunocytochemistry for quantification of skin changes by heat evil; melanin analysis, catalase assay or DCFH-DA (dichlorofluorescin diacetate assay) for quantification of skin changes caused by dampness evil; immunocytochemistry for quantification of skin changes by dry evil; and DAPI staining or β-gal staining for quantification of skin changes caused by fire evil. Such analysis methods have a common characteristic in that they include the analysis of cytokines, the analysis of antioxidant activity, the analysis of cytotoxicity, the analysis of cell proliferation, and the analysis of patterns of variations in specific proteins and genes. Also, in the present invention, skin changes can be quantified by measuring cell survival rate, cell growth rate, cell membrane damage, DNA damage and the change in the expression in specific proteins using MTT assay, LDH assay, comet assay, and Western bolt analysis employing specific antibodies, which can measure cellular changes.

Cells which can be used in the present invention are all skin cells, which can use single-layer cultures and 3D tissue cultures, including keratinocytes which can be primarily isolated and cultured from human and animal skins. Furthermore, macrophage cell lines such as RAW 264.7 cells, which are attributable to the immunity of the skin, may also be used. In addition, human biopsy tissues may also be used.

Finally, the suitability of the above methods selected as typical medicinal herbs that defend the skin from the six external evils is confirmed.

In this step, a significant difference between a control group and a test group can be measured by determining if those selected as stimuli of the six external evils and as measurement indices enable the effects of medicinal herbs to be confirmed.

Medicinal herbs which can be used in the present invention include not only those having the properties and characteristics of conventional medicinal herbs, but also prescriptions made on the basis of Chinese traditional medicinal books such as Donguibogam and Sanghanrhon (Theory of Typhoid), or prescriptions made based on Oriental medical diagnosis by Oriental medical doctors. More specific examples of the medicinal herbs include, but are not limited to, Angelica dahurica, Zingiberis officinale, Lonicera japonica, Scutellaria baicalensis, Rehmannia glutinosa and Anemarrhena asphodeloides.

Also, the present invention relates to a method of screening a skin condition-improving material using the above-described method for quantifying skin changes caused by the six external evils.

The inventive method for screening a skin condition-improving material comprises the steps of:
1) adding a candidate to cells and stimulating the candidate-added cells with at least one stimulus selected from among six external evils, including wind, cold, heat, dampness, dryness and fire evils;
2) treating cells with a medicinal herb as a positive control group together with the candidate before or after the stimulation of step 1);
3) measuring and quantifying the condition of the skin by a cellular biochemical method; and
4) comparing quantitative values obtained for the candidate with quantitative values obtained for the medicinal herb as the positive control group to determine the effect of the candidate.

The cultured cells, the stimuli of the six external evils, the cellular biochemical methods, and the medicinal herbs used as the positive control group, which are used in the above-described screening method may be the same as those used in the above-described method of quantifying skin changes caused by the six external evils.

This screening method may be broadly applied not only to a single cell culture system, but also to the human skin.

### MODE FOR THE INVENTION

Hereinafter, the present invention will be described in further detail with reference to examples and test examples. However, a person skilled in the art will appreciate that these examples are not to be construed to limit the scope of the present invention and that various modifications, substitutions and additions may be made without departing from the scope of the present invention.

### REFERENCE EXAMPLE 1

### Preparation of Medicinal Herb Extracts

1 kg of each of Angelica dahurica, Zingiberis officinale, Lonicera japonica, Scutellaria baicalensis, Rehmannia glutinosa and Anemarrhena asphodeloides was extracted with 5 l of 70% ethanol for 3 hours, and then filtered. The remaining filtrate was concentrated under reduced pressure, thus obtaining a 70% ethanol extract of each of the medicinal herbs. These samples were used to carry out the following experiments.

### EXAMPLE 1

### Method for Quantifying Skin Changes Caused by Wind Evil

Human keratinocyte HaCaT cells were cultured in 10% FBS-DMEM under conditions of 37° C. and 5% CO₂. The cells were seeded into a 6-well cell culture plate at a density of 5×10⁵ cells/well and treated with 1 μg/ml of LPS to induce wind evil. The wind evil-induced test group was treated with 10 μg/ml of the medicinal herb Angelica dahurica extract (Reference Example 1) having the properties of expelling wind, relieving exterior syndromes and expelling wind-dampness, and the effects of treatment with the medicinal herb extract were examined. Cells cultured without treatment with the LPS and the Angelica dahurica extract were used as a control group, and skin changes caused by the wind evil were quantified by measuring the changes in expression and activity of COX-2 (cyclooxygenase-2) protein relative to the control group.

On the day following the day of treatment with the test material, the sample was collected and the disrupted cell solution was developed through 10% SDS-gel electrophoresis and transferred to a nitrocellulose membrane, which was then blocked with 5% non-fat milk. The blocked nitrocellulose membrane was analyzed by Western blot using COX-2 monoclonal antibody, and the expression of COX-2 protein playing an important role in inflammatory reactions was measured by densitometer. The measurement results are shown in Table 1 below.

TABLE 1
Increase in COX-2 expression caused by LPS used as
wind stimulus and the effect of Angelica dahurica extract

| | Treatment of | Degree of | |
|---|---|---|---|
| LPS | Angelica | Expression | Increase rate (%) of |
| Treatment | dahurica extract | of COX-2 (AU) | Expression of COX-2 |
| − | − | 723 | 0.0 |
| + | − | 1571 | 117.3 |
| + | + | 1218 | 68.5 |

As can be seen from the results in Table 1, the Angelica dahurica extract showed the effect of inhibiting the LPS-induced inflammation by about 40%. This suggests that an inflammation-inducing factor such as LPS can be used as the wind stimuli and that a protein such as COX-2 can be used as an index for measuring cellular changes.

### EXAMPLE 2

### Method of Quantifying Skin Changes Caused by Cold Evil

Human keratinocyte HaCaT cells were cultured in 10% FBS-DMEM under conditions of 37° C. and 5% CO₂. The cells were seeded into a 3.5-well cell culture dish at a density of 2×10⁵ cells/well and incubated at −15° C. to induce cold evil. The cold evil-induced test group was treated with 10 μg/ml of the medicinal herb Zingiberis officinale extract (Reference Example 1) having the property of expelling cold while maintaining warm, and the effects of treatment with the medicinal herb were examined. Cells cultured without low-temperature treatment and treatment with the Zingiberis officinale extract were used as a control group, and skin changes caused by the cold evil were quantified by measuring the change in cell growth rate relative to the control using MMT analysis.

On the day following the day of low-temperature treatment and treatment with the test material, the cells being cultured were treated with 0.5 μg/ml of MMT reagent (3-(4,5-dimethyl-2-thiazolyl)-2,5-diphenyl-2H-tetrazolium bromide, Sigma) for 2 hours, and the culture medium was removed. The remaining purple precipitate was dissolved in DMSO, and then measured for absorbance at 560 nm. The measurement results are shown in Table 2 below.

TABLE 2
Decrease in cell growth rate after low-temperature treatment
with cold stimulus and the effect of Zingiberis officinale extract

| Low- | Treatment of | | |
|---|---|---|---|
| Temperature | Zingiberis officinale | | |
| Treatment | extract | OD₅₆₀ (AU) | Cell Growth Rate (%) |
| − | − | 0.9679 | 100.0 |
| + | − | 0.4825 | 49.9 |
| + | + | 0.7489 | 77.4 |

As can be seen from the results in Table 2, the Zingiberis officinale extract effectively restore the decrease in cell growth rate caused by low-temperature treatment. This suggests that low-temperature treatment can be used as the cold stimulus and that cell growth rate can be used as an index for measuring cellular changes.

### EXAMPLE 3

### Method for Quantifying Skin Changes Caused by Heat

Human kerationcyte HaCaT cells were cultured in 10% FBS-DMEM under conditions of 37° C. and 5% CO₂. The cells were attached to a 3.5-well cell culture dish at a density of 2×10⁵ cells/well and incubated at 44° C. to induce heat evil. The heat evil-induced test group was treated with 10 μg/ml of the medicinal herb Lonicera japonica extract (Reference Example 1) having the property of clearing heat and relieving toxicity, and the effects of treatment with the medicinal herb extract were examined. The results of quantification of skin changes caused by heat were obtained by measuring cell membrane stability using LDH assay (Sigma Aldrich, Product Number TOX-7). On the day following the day of high-temperature and treatment with the medicinal herb extract, the cells being cultured were collected and centrifuged at 12000 rpm for 3 minutes such that the cell debris was completely precipitated, and only the supernatant was collected. 100 μl of the supernatant was added to a 98-well plate, and an LDH assay mixture (LDH assay substrate: cofactor: dye solution=1:1:1) was made immediately before use and mixed with the supernatant in an amount of 200 μl. The resulting mixture was allowed to react for 20 minutes while blocking light, and 25 μl of 1N HCl solution was added thereto to terminate the reaction. Then, the reaction material was measured for absorbance at 490 nm and 690 nm. The absorbance at 690 nm was subtracted from the absorbance at 490 nm to remove the background, thus correcting the measured absorbance value, and the measurement results are shown in Table 3. Cell membrane damage rate was calculated based on the control group treated with Lonicera japonica extract without high-temperature treatment.

TABLE 3
Increase in cell membrane damage after high-temperature treatment
with heat stimulus and the effect of Lonicera japonica extract

| High- | Treatment of | | Cell Membrane |
|---|---|---|---|
| Temperature | Lonicera japonica | Corrected D₄₉₀ | Damage |
| Treatment | extract | (AU) | Rate (%) |
| − | − | 0.1916 | 100.0 |
| + | − | 0.3577 | 186.7 |
| + | + | 0.3113 | 162.5 |

As can be seen from the results in Table 3, the Lonicera japonica extract could effectively reduce the cell membrane damage rate caused by high-temperature treatment. This suggests that high-temperature treatment can be used as the stimulus of heat and that cell membrane damage rate can be used as an index for measuring cellular changes.

### EXAMPLE 4

### Method for Quantifying Skin Changes Caused by Dampness Evil

Human keratinocyte HaCaT cells were incubated in 10% FBS-DMEM under conditions of 37° C. and 5% CO₂. The cells were seeded onto a 98-well cell culture plate at a density of 1×10⁵ cells/well and treated with 1 mM H₂O₂ to induce dampness evil. The dampness evil-induced test group was treated with 10 μg/ml of the medicinal herb Scutellaria baicalensis extract (Reference Example 1) having the property of clearing heat and drying dampness, and the effects of treatment with the medicinal herb tract were examined.

The results of quantification of skin changes caused by dampness evil were obtained by measuring cytotoxicity using MTT assay. On the day following the day of treatment with H₂O₂ and the medicinal herb extract, the cells being cultured were treated with 0.5 μg/ml of MTT reagent (3-(4,5-dimethyl-2-thiazolyl)-2,5-diphenyl-2H-tetrazolium bromide, Sigma) for 2 hours, and then the culture medium was removed. The remaining purple precipitate was dissolved in DMSO, and then measured for absorbance at 560 nm. The measurement results are shown in Table 4 below. Cell survival rate was calculated based on the control group not treated with H₂O₂ and the Scutellaria baicalensis extract.

TABLE 4
Increase in cytotoxicity after H₂O₂ as dampness
stimulus and the effect of Scutellaria baicalensis extract

| | Treatment of | | |
|---|---|---|---|
| H₂O₂ | Scutellaria | | Cell Survival Rate |
| Treatment | baicalensis extract | OD₅₆₀ (AU) | (%) |
| − | − | 1.7679 | 100.0 |
| + | − | 0.7789 | 44.1 |
| + | + | 1.426 | 80.7 |

As can be seen from the results in Table 4, the Scutellaria baicalensis extract effectively inhibited the increase in cytotoxicity caused by treatment with H₂O₂. This suggests that H₂O₂ treatment can be used as the dampness stimulus and that cytotoxicity can be used as an index for measuring cellular changes.

### EXAMPLE 5

### Method for Quantifying Skin Changes Caused by Dry Evil

Human keratinocyte HaCaT cells were cultured in 10% FBS-DMEM under 37° C. and 5% CO₂. The cells were seeded onto a 48-well cell culture plate and incubated in a clean bench in the state in which the lid of the culture plate was open, thereby inducing dry evil. The internal conditions of the clean bench inducing the dampness evil were set at a temperature of 29° C. and a humidity of 45%, but these conditions could be changed depending on experimental conditions. While the cells were exposed to dampness conditions, the cells were treated with 10 μg/ml of the medicinal herb Rehmannia glutinosa extract (Reference Example 1) having the property of nourishing Yin to produce body fluid, and the effects of treatment with the medicinal herb extract were examined.

The results of quantification of skin changes caused by dryness were obtained by measuring cytotoxicity using MTT assay. On the day following the day of dampness treatment and treatment with the medicinal herb extract, the cells being cultured were treated with 0.5 μg/ml of MTT reagent (3-(4,5-dimethyl-2-thiazolyl)-2,5-diphenyl-2H-tetrazolium bromide, Sigma) for 2 hours, and then the culture medium was removed. The remaining purple precipitate was dissolved in DMSO, and then measured for absorbance at 560 nm. The measurement results are shown in Table 5 below. Relative cell survival rates were calculated based on the control group which was neither exposed to dryness nor treated with the Rehmannia glutinosa extract.

TABLE 5
Increase in cytotoxicity after exposure to dry evil
and the effect of Rehmannia glutinosa extract

| Dry | Treatment of | | |
|---|---|---|---|
| Exposure | Rehmannia glutinosa | | Cell Survival Rate |
| Treatment | extract | OD₅₆₀ (AU) | (%) |
| − | − | 1.463 | 100.0 |
| + | − | 0.6789 | 46.4 |
| + | + | 0.9127 | 62.4 |

As can be seen from the results in Table 5, the Rehmannia glutinosa extract effectively inhibited the increase in cytotoxicity caused by exposure to dryness. This suggests that exposure to dryness can be used as the dry stimulus and that cytotoxicity can be used as an index for measuring cellular changes.

### EXAMPLE 6

### Method for Quantifying Skin Changes Caused by Fire Evil

Human keratinocyte HaCaT cells were cultured in 10% FBS-DMEM under conditions of 37° C. and 5% CO₂. The cells were seeded into a 5-well cell culture plate at a density of 5×10⁵ cells/well and irradiated with UVB to induce fire evil. The dose of UVB irradiation inducing fire evil was 40 mJ/cm², but it could be changed depending on experimental conditions. While the cells were irradiated with UVB, the cells were treated with 10 μg/ml of the medicinal herb Anemarrhena asphodeloides extract (Reference Example 1) having the property of clearing heat and purging fire, and the effects of the medicinal herb extract were examined.

The results of quantification of skin changes caused by fire evil were obtained by measuring the degree of DNA damage using comet assay. On the day following the day of UV irradiation and treatment with the medicinal herb extract, the cells being cultured were collected, and 20 μl of the collected cells were added to 200 μl of molten LMagarose. Then, 75 μl of the mixture was immediately transferred to a comet slide. Then, comet slide was immersed sequentially in a lysis solution and a newly prepared alkaline solution (0.6 g NaOH/50 ml DIW) and electrophoresed. 50 μl of SYBR green I dilution was dropped onto the slide to stain DNA, and then, the slide was observed and photographed with a fluorescence microscope at 494 nm/512 nm filters to compare the DNA tail length between the control group and the test group. The comparison results are shown in Table 6 below. Relative DNA damage rates were calculated based on the control group which was neither exposed to UVB nor treated with the Anemarrhena asphodeloides extract.

TABLE 6
Increase in cytotoxicity after irradiation with UVB as fire
evil and the effect of Anemarrhena asphodeloides extract

| | Treatment of | | |
|---|---|---|---|
| UV light | Anemarrhena | Tail Length | DNA damage rate |
| irradiation | asphodeloides extract | (μm) | (%) |
| − | − | 65.4 | 100.0 |
| + | − | 112.4 | 171.9 |
| + | + | 78.2 | 119.6 |

As can be seen from the results in Table 6, the Anemarrhena asphodeloides extract effectively inhibited the increase in DNA damage rate caused by UV light irradiation. This suggests that UV light irradiation can be used as the stimulus of fire and that DNA damage can be used as an index for measuring cellular changes.

The above-described Examples illustrate typical examples of the present invention and may be modified in any form. For example, in the case of wind evil, Dermatophagoides farina fragments may be used instead of LPS as the wind stimulus. In the case of cold, heat and dry evils, the temperature and humidity conditions are not limited to the conditions specified in the above Examples and can be selected from a wide range of conditions, including temperatures higher or lower than the skin temperature and a wide range of humidity conditions. In the case of dampness evil, a material inducing the production of ROS may be used instead of H₂O₂ as the dry stimulus, and in the case of fire evil, UVA or IR light may also be used as the fire stimulus.

We claim:

1. A method for quantifying skin changes caused by heat evil, the method comprising the steps of:
(a) selecting a skin stimulus (i.e., heat or heat temperature) for the heat evil from temperature ranging from 39° C. to 50° C.;
(b) selecting a suitable cellular biochemical method, which can measure skin cell changes, according to the selected skin stimulus, wherein the suitable cellular biochemical method is selected from the group consisting of TNF-α ELISA, PGE₂ ELISA, interleukin expression analysis, cell proliferation assay, FAS analysis, RT-PCR, immunocytochemistry, melanin analysis, catalase assay, dichlorofluorescin diacetate assay (DCFH-DA), DAPI staining, β-gal staining, MTT assay, LDH assay, comet assay, and Western blot analysis employing specific antibodies;
(c) measuring condition of the skin of a subject by the selected cellular biochemical method;
(d) stimulating the skin with the selected skin stimulus, and then quantifying the degree of cellular changes according to the intensity of the selected heat temperature; and
(e) applying an extract of Lonicera japonica to the skin, and then measuring and quantifying the conditions of the skin by the selected cellular biochemical method.

2. The method according to claim 1, wherein the index for quantifying the degree of cellular changes caused by the skin stimulus for the heat evil is cell membrane damage rate of human keratinocyte HaCaT cells.

3. A method for screening a skin condition-improving material, the method comprising the steps of:
(a) adding an unknown herbal extract, which is suspected of having skin condition-improving properties, as a candidate, to cells and stimulating the unknown herbal extract-added cells with a skin stimulus (i.e., heat or heat temperature) for the heat evil from temperature ranging from 39° C. to 50° C.;
(b) treating cells with a known herb extract as a positive control group together with the unknown herbal extract before or after the stimulation of step (a), wherein the known herbal extract is an extract from Lonicera japonica;
(c) measuring and quantifying condition of the skin by a cellular biochemical method, wherein the cellular biochemical method is selected from the group consisting of TNF-α ELISA, PGE₂ ELISA, interleukin expression analysis, cell proliferation assay, FAS analysis, RT-PCR, immunocytochemistry, melanin analysis, catalase assay, dichlorofluorescin diacetate assay (DCFH-DA), DAPI staining, β-gal staining, MTT assay, LDH assay, comet assay, and Western blot analysis employing specific antibodies; and
(d) comparing quantitative values obtained for the unknown herbal extract with quantitative values obtained for the known herb extract to determine the effect of the unknown herbal extract.

4. The method according to claim 3, wherein the index for quantifying the degree of cellular changes caused by the skin stimulus for the heat evil is cell membrane damage rate of human keratinocyte HaCaT cells.

5. The method according to claim 3, wherein the skin condition-improving material is a material having the effect of improving skin aging, pigmentation, skin dryness, or skin troubles caused by the heat evil.
